(19) United States
(12) Patent Application Publication
(10) Pub. No.: US 2010/0100030 A1
(43) Pub. Date: Apr. 22, 2010

(54) Microbe Reductions with Photosensitizers

(75) Inventors: Tim Driscoll, Hokyoke, MA (US); Verónica Inés Mega, Buenos Aires (AR); Wolfgang Neuberger, Dubai (AE)

Correspondence Address: BOLESH J. SKUTNIK;CERAMOPTEC INDUSTRIES, INC., 515 SHAKER RD., EAST LONGMEADOW, MA, 01028, US

(73) Assignee: CeramOptec Industries Inc.

(21) Appl. No.: 12/643,467

(22) Filed: Dec. 21, 2009

Related U.S. Application Data
(63) Continuation-in-part of application No. 11/523,203, filed on Sep. 19, 2006.
Application No. 11/523,203 is a continuation-in-part of application No. 10/241,958, filed on Sep. 12, 2002, now abandoned.

(51) Int. Cl. A61M 37/00; A61K 9/14; A61K 31/195; A61N 5/06

(52) U.S. Cl. 604/20; 424/489; 514/561; 977/906

(57) ABSTRACT

Methods, devices and composition are disclosed for treating oral, skin and nail diseases, including inflammatory periodontal disease, onychomycosis and dermatophytosis. In a preferred embodiment a method for treating oral, skin and nail infections comprises administering a photosensitizing compound in long term effect or timed release formulations, including local highly concentrated formulations, and activating the photosensitizer with radiation to selectively destroy bacteria, fungi and other microbial bodies. In another preferred embodiment, photosensitizers are housed within nanoparticles, and can be gradually released through biodegradation or periodically released by such processes as brushing, irradiation and chemically induced release. The diffusion speed of the photosensitizers can be accelerated by brushing. In another embodiment, the photosensitizer molecule is also modified to more effectively target the molecule to the unwanted oral bacterial and fungal species. In another embodiment devices for treating oral, skin and nail infections includes a laser radiation source and delivery apparatuses such as brush or teethed comb for nail and scalp infections respectively.

### REFERENCE TO A RELATED CASE

This application is a continuation-in-part of co-pending U.S. patent application Ser. No. 11/523,203 filed on Sep. 19, 2006 by Wolfgang Neuberger, inventor, entitled “TREATMENT OF PERIODONTAL DISEASE WITH PHOTOSENSITIZERS”, which in turn is a continuation-in-part of U.S. patent application Ser. No. 10/241,958 filed on Sep. 12, 2002 by Wolfgang Neuberger, inventor, entitled “TREATMENT OF PERIODONTAL DISEASE WITH PHOTOSENSITIZERS” and incorporated by reference herein.

### BACKGROUND OF THE INVENTION

1. Field of the Invention

The present invention relates to the use of photosensitizing compounds in long term effect or timed release formulations and laser irradiation to kill the microbes involved in a number of oral, skin and nail microbial infection diseases, such as bacteria, viruses, yeasts and fungi. More particularly, present invention provides methods, compositions and devices to treat oral bacteria and dermatophytosis such as Tinea barbae, Tinea capitis, Tinea cruris and Tinea unguium caused by pathogenic fungi.

2. Information Disclosure Statement

Advanced periodontal disease is one of a large number of oral infectious diseases, and is the principal cause of tooth loss in those over 30 years old. The term “periodontal disease” actually encompasses a number of diseases that affect the supporting tissues of the teeth. Gingivitis, an inflammation of the gingival tissue, may be the first sign of oncoming periodontal disease and most, if not all, periodontal disease features accompanying gingivitis. Another example is periodontitis, defined as inflammation involving the gingival unit, periodontal ligament, alveolar bone, and cementum which is characterized by loss of clinical attachment of the gingiva and radiographic loss of bone.

The vast body of evidence indicates that the primary cause of both gingivitis and periodontitis is bacterial activity. Bacteria attach to the tooth surface and gingival margin colonize and form an organized mass that is referred to as bacterial plaque. This plaque, if allowed to remain, brings about inflammatory changes in the tissues. The reaction of the host tissues to this bacterial attack is through an inflammatory and immunologic defense mechanism. Periodontal diseases arise from the interaction between bacterial cells and their products in dental plaque and the host defense mechanisms.

Along with teeth, nails are an appendage of the skin. They are horn-like structures at the end the finger or toe, made of keratin. When microbes like fungus infect the nail, a disease commonly known as onychomycosis, they may cause discoloration, thickening, brittleness, crumbling, shape distortions and dullness.

As in periodontal disease, this medical disorder can also become a cosmetic problem that alters social healthiness. Whereas in general in the first case noticeably unpleasant odors are exhaled in breathing producing social discomfort, in the case of toenail the sick is generally embarrassed to go barefoot in public so stays away from the beach or swimming pools or avoid wearing sandals or open-toed shoes. In some conditions, infected finger or toe nails may also separate from the nail bed, a condition called onycholysis, causing pain and a slightly foul odor. Unfortunately, many risk factors are associated with finger and toe nail fungus including increasing age, nail trauma, sweaty feet, poor circulation, poor hygiene, foot fungus and a compromised immune system. Furthermore, the condition can get worse as nail fungus can spread to other nails or the skin, and can be accompanied by a secondary bacterial or yeast infection in the nail bed which can cause permanent damage if left untreated.

A variety of treatment modalities have been utilized to combat periodontal disease and prevent its progression including periodontal surgery and chemotherapy. However, these conventional methods described above suffer from a number of significant drawbacks, causing sensitivity and resulting in poor aesthetics by lengthening the clinical crown and altering the gingival contours.

Likewise, finger an toe nail fungus can be difficult to treat as treatment failures and recurrences are common, seeming a never-ending problem. Furthermore, nails require between 12 to 18 months to growth, depending on age, gender, season, exercise level, diet and hereditary factors. Thus, treatment effectiveness is essential as the end result of treatment will only be seen after the nail grows back completely. Different treatments have been proposed including antifungal oral medications, nail creams, ointments and lacquers, but many of them have proved to be ineffective or produced considerable side effects ranging from skin rashes to liver damage or may interact with other commonly used drugs. Among the antifungal oral medications used to treat nail fungus, Itraconazole, Fluconazole, Ketoconazole, Griseofulvin and Terbinafine are the most typical drugs prescribed. These medications help a new nail grow free of infection, slowly replacing the infected portion of your nail. Due to the extended period of time required for these drug treatments, blood tests are regularly needed to check the patient's liver function during the course of therapy.

In some severe infections or following recurrence, removal of the nail is prescribed. A new nail will usually grow in its place taking as long as a year to grow back completely, though it may grow with abnormal shape or appearance. In addition, it is a painful treatment which usually renders the nail susceptible to infections requiring a regular application of antibiotics.

Current therapies to treat bacteria and fungus in oral cavity or nails are associated with sensitivity, poor aesthetic results, treatment failures and recurrences. However, another alternative, known as photodynamic therapy (PDT) has been proposed as an attractive method of eliminating oral bacteria and bacteria in topical and gastrointestinal infections because these sites are relatively accessible to illumination. For example, Wilson and Harvey proposed in U.S. Pat. No. 5,611,793 the use of photosensitizers in combination with suitable irradiation to treat several oral diseases. However, this method suffers from the fact that the duration of the treatment is not sufficient. To treat periodontitis, effective concentration of the active substance in the periodontal pocket must be maintained for a relatively long time.

Considering nail fungus, there are many species of fungi that can affect nails. Trichophyton rubrum is one of the most common of the dermatophytes with the tendency to infect the toenail; but not only fungi can infect nails, there is also a type of onychomycosis caused by yeast called Candida which may be the most common cause of fungal fingernails. International Publication WO 2004/069273 by Smijs et al. discloses a PDT treatment involving the use of a photosensitizer compound for the treatment of skin-borne fungus such as Trichophyton rubrum. The method comprises the application of a porphyrin compound with a specified structural formula and the illumination of the location where the pharmaceutical composition was applied with light having a wavelength which can be absorbed by the compound in the presence of oxygen. The method of preparing the pharmaceutical composition requires the addition of a substance to make skin and/or nail more permeable to the compound.

Another example of treating an area of skin or nail affected with a pathogen is disclosed in U.S. Pat. No. 6,090,788 and International Publication No. WO 99/04628 by Lurie. The method comprises the step of causing a fungal pathogen to include a light absorbing substance via ultraviolet irradiation, by subjecting the fungal pathogen to a pigment or to a dextrose compound. Then, the area of skin or nail is irradiated with a light beam having at least one wavelength absorbable by the light absorbing substance, selected such that an interaction between the light beam and the light absorbing substance results in excessive heating, which, by itself, is sufficient for destruction of the fungal pathogen. Subsequently the area is treated with an anti-irritant. Care has to be taken with excessive heat in the treated area, as severe and irreversible damage can occur.

Intending to treat dermatophytic fungal infections with PDT, International Publication No. WO 2008/109424 by Loebel et al. discloses a composition for treating fungal infections comprising a photosensitizer, an effective amount of antifungal agent, and a pharmaceutically acceptable delivery system to be applied to a locus and irradiating the locus with a light source at a wavelength absorbed by the photosensitizer so as to destroy the microbes in it. The method further comprises the reduction of the nail's thickness via electromagnetic ablation and the creation of micro-channels between top and bottom of the nail prior to applying the antifungal composition, comprising a time-consuming and unpleasant treatment.

Alternatively, the application of thermal energy or radiation source for treating diseased nails without the use of a dying agent or an exogenous chromophore is disclosed in Patent Application Publication No. US 2006/0212098 by Demetriou et al. The energy source is applied to the target area to thermally deactivate an unwanted organism such as a bacterium, mold, fungus, parasite or virus without causing substantial unwanted injury to at least one of the nail bed and the nail plate. The method further comprises the introduction of an index matching solution into a porous region of the diseased nail prior to delivering radiation such as mineral oil, glycerin, glycol or water.

Thus, there is a need to effectively cope with extremely difficult-to-treat pathogenic microbial diseases such as onychomycosis, dermatophytic infections, gingivitis and periodontitis. It would be useful to provide formulations and a method of treatment for pathogenic microbial diseases which overcomes the drawbacks of prior art methods and further may be practiced or continued by patients at home as well as utilized by professional practitioners.

The present invention addresses this need.

### BRIEF SUMMARY AND OBJECTIVES OF THE INVENTION

It is an objective of the present invention to provide a method for treatment of oral, skin and nail diseases characterized by long duration from a single dose/application.

It is another objective of the present invention to use photodynamic therapy (PDT) for the treatment of oral, skin and nail diseases with photosensitizers applied in a timed release formulation or coating at the desired sites.

It is a further objective of the present invention to modify the photosensitizer to more effectively target the photosensitizer to the unwanted (bacterial and fungal) species.

It is a still further objective of the present invention to provide devices emitting suitable wavelengths to activate the photosensitizers by irradiation, applied either by a health professional or as repeated treatment by the patient at home e.g. using a laser brush or a lamp.

Briefly stated, the present invention discloses methods, devices and compositions for treating oral, skin and nail diseases, including inflammatory periodontal disease, onychomycosis and dermatophytosis. In a preferred embodiment a method for treating oral, skin and nail infections comprises administering a photosensitizing compound in long term effect or timed release formulations, including local highly concentrated formulations, and activating the photosensitizer with radiation to selectively destroy bacteria, fungi and other microbial bodies. In another preferred embodiment, photosensitizers are housed within micro- or nanoparticles, and can be gradually released through biodegradation of the particles or periodically released from the particles by such processes as brushing, irradiation and chemically induced release. The diffusion speed of the photosensitizers can be accelerated by brushing. In another embodiment, the photosensitizer molecule is also modified to more effectively target the molecule to the unwanted oral bacterial and fungal species. In another embodiment devices for treating oral, skin and nail infections includes a laser radiation source and delivery apparatuses such as brush or teethed comb for nail and scalp infections respectively.

The above, and other objectives, features and advantages of the present invention will become apparent from the following description.

### BRIEF DESCRIPTION OF FIGURES

FIG. 1a shows a side view of one embodiment depicting a delivery apparatus to treat fungal infections in toe and finger nails.

FIG. 1b depicts a front view of delivery apparatus to treat fungal infections in toe and finger nails.

FIG. 2a depicts a side view of one embodiment of delivery apparatus to treat fungal infections in the scalp.

FIG. 2b shows a frontal view of delivery apparatus to treat fungal infections in the scalp.

FIG. 3 illustrates one embodiment of a method for fungal reduction in contaminated toenail.

FIG. 4 shows another embodiment of a method for disinfection or sterilization of manicure and pedicure tools.

### DETAILED DESCRIPTION OF PREFERRED EMBODIMENTS

The present invention provides methods, composition and devices for disinfecting or sterilizing tissues, a wound or lesion in the oral cavity or skin, an infected nail or a contaminated area of the skin. In a preferred embodiment, a method comprises applying a photosensitizing compound in a timed release formulation, as well as a formulation which provides locally high concentrations of primary protected photosensitizers, to infected areas of the skin, nail, tissue, wound or lesion. Photosensitizers that produce highly reactive singlet oxygen upon activation by irradiation are known to be effective in destroying unwanted (bacterial) species and thereby disinfecting and sterilizing lesions and wounds as well as implants in the oral cavity. Additionally, they have the potential to effectively destroy unwanted microbial species such as fungi, viruses and yeast and thereby disinfecting and sterilizing contaminated areas of the nail and skin. The timed release is characterized by providing a sustained concentration of the photosensitizer in or around a treatment area during a prolonged time period in which the photosensitizer can be activated repeatedly at predetermined intervals two or several times by irradiating the tissue, wound or lesion with a light source at a wavelength absorbed by the photosensitizing compound. To prevent unwanted photosensitizer activation, between irradiations treatment site is covered with dark clothes, gloves, close shoes or caps.

This timed release formulation is characterized by its ability to maintain a certain concentration of the photosensitizer over a prolonged period of time in the treatment site, and it thus provides the ability to repeatedly activate the photosensitizing compound after a single application and thereby achieve healing due to repeated treatments over an extended time period with only one application of the formulation. An alternative description of this action is that only a portion of the photosensitizer is available for activation by a first irradiation of treatment site. In one embodiment, a long term effect is achieved by administering the photosensitizer bound to particles or molecules which release the drug slowly, or by coating the respective surfaces with a specially modified dye. In another embodiment, the photosensitive molecules are included in and released from micro- or nanoparticles by controlled diffusion. Biodegradable nanoparticles can be used to avoid the step of actively releasing the molecules. In that case, the duration of the timed release would be limited and would vary depending on the composition of the nanoparticles. In another embodiment, release from some or all of the micro- or nanoparticles can be actively and controllably induced by the user or practitioner as part of the treatment.

The administration of present composition for treatment of nail and skin microbial diseases comprising a photosensitizer in a timed release formulation includes, but it is not limited to direct application, application with a gel, application with a lacquer, application with a cream, application with a ointment, application with a sprayer, application with a lotion and combinations of them.

These formulations are especially useful in treating chronic diseases where repeated treatments are necessary to achieve healing. After a photosensitizer in a timed release formulation is administered to the affected area, it is left in contact with the microbes for a period of time to enable the microbes to take up some of the photosensitizer and become sensitive to the light radiation. The photosensitizers are subsequently activated by radiation at suitable wavelengths for one or more treatments to destroy the target microbes. In some cases photosensitizer formulation may be used in a light-independent manner, exposing the treated area of skin or nail to environmental radiation to treat or prevent microbial infection. The activation can be carried out by a health professional or even by the patient at home with, for example, a laser brush or a lamp, operating at a wavelength which activates the photosensitizer. Additionally, irradiation efficiency may be increased by using radiation sources with more than one radiation wavelength, one to activate the photosensitizing formulation administered exogenously and another to activate any endogenously light absorbing substances present in the microbes or treated tissue. In any case, suitable irradiation parameters such as time, energy and power density are adjusted according to patient's disease condition.

In preferred aspects of the invention, the described treatment methods with photosensitizing compounds and light irradiation can be applied to a variety of tasks including:

the destruction of disease-related microbes such as fungi and yeast in the nail in order to treat or prevent chronic onychomycosis and the like;

the destruction of disease-related microbes such as fungi and yeast in a contaminated area of the skin in order to treat or prevent skin-borne diseases including chronic dermatophytosis and the like; and

the disinfection or sterilization of manicure and pedicure tools and the like used for cosmetic beauty treatments.

The advantage of the use of photosensitive compounds over other methods, such as antibiotic treatments, is the ability to controllably activate the compound with radiation and therefore enhance treatment effects. The long term use of antibiotics often promotes the development of resistance rendering the agents clinically ineffective. Additionally, high concentrations of antibiotics may have undesired side effects in the tissue and on the oral microflora. Since the photosensitive compound shows no toxicity without irradiation, the application of high concentrations and short and possibly repeated activation times show enhanced therapeutic effects and reduced side effects. High concentrations of the photosensitizer can be achieved, for example, by the use of specialized micro- and nano-particles such as dendrimers. Moreover, the ability of the compound to destroy only the unwanted species and leave healthy tissue intact can be enhanced by modifying the compound with targeting moieties. These modifications can affect chemical or physical properties that show an affinity to areas such as the cell membranes of unwanted fungal/bacterial species. Other targeting methods including coupling with antibodies or other affinity molecules are conceivable.

It is preferred that in the method of the present invention comprising irradiation and exogenous substance administration, the photosensitizers used are activatable by light at the red end of the visible spectrum or at longer wavelengths, preferably between 630 and 800 nm. Several “second generation” photosensitive compounds with these spectral absorption properties are known to date, and include porphyrins, chlorins, pheophorbides, bacteriopheophorbides, phthalocyanines, naphthalocyanines, thiazines, xanthenes, pyrrylium dyes, psoralens, quinones. Other compounds may also be used with the present invention, including photosensitizer precursors such as aminolevulinic acid (ALA), which naturally in a mammalian body converts to a photosensitizer, protoporphyrin IX.

Light at these wavelengths is better able to penetrate tissues surrounding a wound, lesion or infected cells, such as oral tissues, nails, skin and, in particular, blood which may be present in the sites to be treated. Light at these wavelengths is also capable of going through the entire nail thickness and reaching the nail bed, activating the delivered photosensitizer in all the treated tissue.

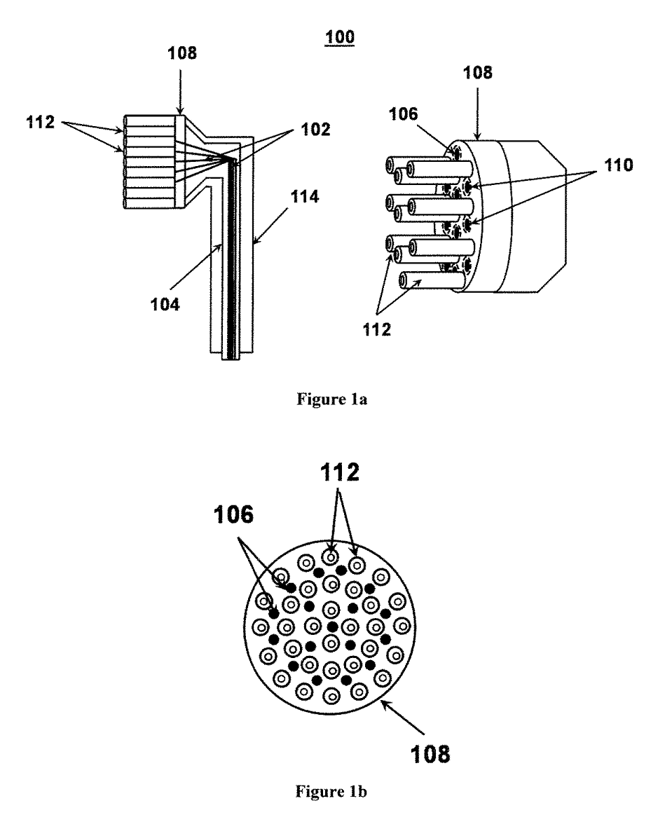

In a preferred embodiment, the present invention provides devices comprising a radiation source and a delivery apparatus, to effectively destroy or inactivate infectious microbe species such as fungi, viruses, bacteria and yeast. Radiation source emits suitable wavelengths to activate by irradiation exogenously administered photosensitizers, inherent light absorbing substances present in the microbes or treated tissues or a combination of them. Radiation source includes coherent and incoherent radiation sources such as laser radiation source, light emitting diodes source and lamp radiation source (incandescent, xenon arc and metal halide lamps) and/or sunlight. In a preferred embodiment, radiation sources emitting radiation of wavelengths suitable to activate photosensitizers deliver pulsed or continuous laser radiation in the range of 630 and 800 nm. Laser radiation energy is distributed with the aid of a delivery apparatus, such as a brush or lamp. Laser irradiation may be applied either by a health professional or as repeated treatment by the patient at home. To treat periodontal diseases the preferred delivery apparatus is like the toothbrush disclosed in U.S. Pat. No. 5,658,148 and U.S. Pat. No. 6,056,548 and incorporated by reference herein. In another embodiment, a delivery apparatus to treat fungal infections in toe and finger nails is depicted in FIG. 1. The brush 100, preferably a plastic brush, contains one or more optical fibers 102 which deliver laser radiation and a conduit 104, for applying extra oxygen in gaseous state, an oxygenated solution and/or a solution containing the photosensitizer to treatment site. Optical fiber tip 106 may be included in brush head 108 and emits laser radiation through openings 110 in brush head 108, at the end of hollow bristles 112 inserted in brush head 108 or in both places. Conduit 104 may also be included in brush head 108 and emits extra oxygen in gaseous state, an oxygenated solution and/or a solution containing the photosensitizer under pressure through openings 110 in brush head 108, at the end of hollow bristles 112 inserted in brush head 108 or in both places. In order to easily operate brush 100, it is fixed to handle 114. Brush 100 is removable from handle 114 so different brush shapes and sizes containing different bristle diameters may be used depending on the treatment site.

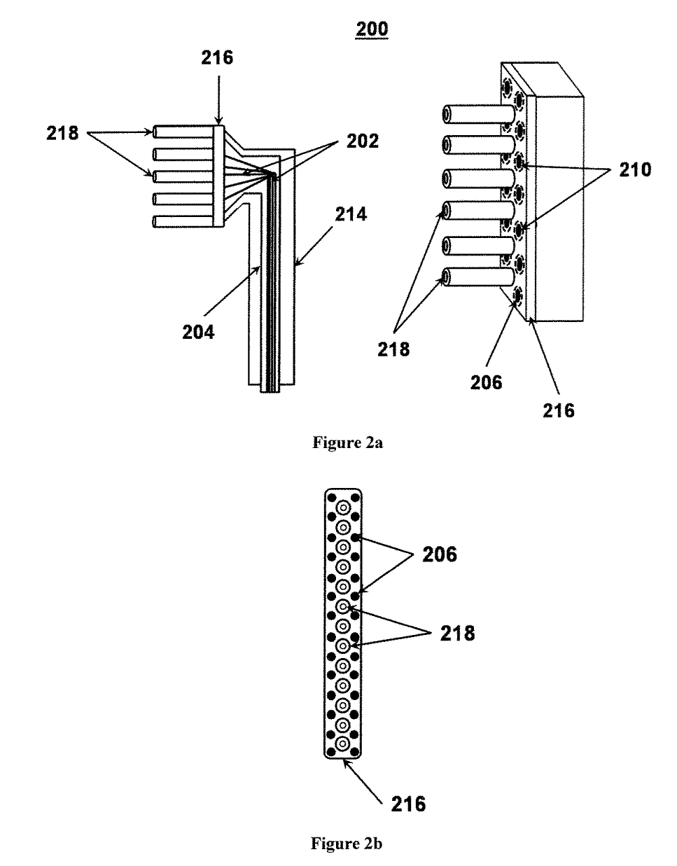

In another embodiment a delivery apparatus to treat fungal infections in the scalp is depicted in FIG. 2. The delivery apparatus is a brush like the one depicted in previous embodiment but with a size that allows effective laser radiation delivery to the treatment site. The delivery apparatus comprises a toothed comb 200, preferably made of plastic, containing one or more optical fibers 202 which deliver laser radiation and a conduit 204 to deliver extra oxygen in gaseous state, an oxygenated solution and/or a solution containing the photosensitizer for enhanced PDT treatment. Optical fiber tip 206 may be included in the edge of the comb 216 and emits laser radiation through openings 210 in the edge of the comb 216, at the end of hollow comb's teeth 218 inserted in the edge of the comb 216 or in both places. Conduit 204 may also be included in the edge of the comb 216 and emits extra oxygen in gaseous state, an oxygenated solution and/or a solution containing the photosensitizer under pressure through openings 210 in the edge of the comb 216, at the end of hollow comb's teeth 218 inserted in the edge of the comb 216 or in both places. In order to easily operate comb 200 it is fixed to handle 214. Comb 200 is removable from handle 214 so different comb shapes and sizes containing different comb's teeth diameters may be used depending on the treatment site.

In a preferred embodiment, the timed release characteristic of the present composition is due to the inclusion of polymeric micro- or nanoparticles, constructed with polymers that contain the photosensitizer molecules. Release of photo sensitizers from these polymers can occur by two general mechanisms. The first mechanism is diffusion resulting in the gradual release of the photosensitive molecules from the implant surface. The second mode of release occurs by the cleavage of the polymer backbone, defined as bulk erosion. Release can also be induced by a number of other processes. Such processes include radiation, ultrasonics, agitation through brushing, and chemical release such as with a solvent.

The micro- or nanoparticles consist of molecules building up polymers that are tolerable and non-toxic to the human or animal body. Several such compositions are known to date, among them are biodegradable and non-biodegradable compositions. Non-biodegradable particles must be removed after a certain time and thus the duration of the deposit effect can be chosen. The life time of biodegradable compounds is limited but can be chosen in a certain time range by varying the composition and size of the polymer. The molecular weight of the polymers has an important effect on the period of drug release and degradation of the microspheres. The products of degradation processes such as hydrolysis of the particles are biologically compatible and metabolizable moieties which are eventually removed from the body. Polymer biodegradation products are formed at a very slow rate, and hence do not affect normal cell function. Moreover, the deposit effect can be achieved by coating the affected areas such as teeth, periodontal pockets, nails and skin with the photosensitizer.

The method of preparation of polymeric nano- or micro-particles may include nail, skin or mucosa chemical penetration enhancers. Due to the different nature of the penetration barriers, appropriate chemical penetration enhancers for the specific tissue to be gone through would be chosen as would do an ordinarily person skilled in the art for standard purely chemically-acting medications. Additionally physical enhancement techniques may be used for improving the photosensitizer formulation delivery such as iontophoresis, electroporation, ultrasound, photomechanical waves, radiofrequency, magnetic fields, micro-needles, jet-propulsion or by any other means.

In addition to the advantage of being able to house and gradually or periodically release photosensitizers, nanoparticles offer an additional benefit. Many photosensitizers suffer from significant inhibition in effectiveness from exposure to saliva, white blood cells, and other natural defenses in the mouth. This especially can prove a significant problem in maintaining an effective concentration of photosensitizers in the mouth for an extended period of time. Because photosensitizers are encased in nanoparticles before treatment, they are shielded from these bodily fluids and thus do not degrade as quickly as they would if left unprotected. Non-biodegradable micro- or nanoparticles serve to protect photosensitizers until the photosensitizers are released. Likewise, biodegradable nanoparticles also protect photosensitizers until they degrade to the point where they release the photosensitizers. In this way, micro- or nanoparticles further serve to prolong the period of time that photosensitizers can remain in the oral cavity.

It should be noted that the photosensitizer has to be in a certain concentration at the site of treatment and, for instance in the treatment of toenail where the treatment site may be scraped with shoes or stockings, it might be necessary to apply the photosensitizers in greater concentration so as to achieve an effective dose. Moreover, the treatment is more effective if the photosensitizer is applied in higher concentrations. These concentrations can be achieved by using highly specialized micro- and nanoparticles like dendrimer-photosensitizer complexes. Since the photosensitive compounds do not show any dark toxicity, these concentrations can be applied without side effects. Because exposure of the surrounding tissues of the oral cavity, nail or infected skin to the activated photosensitizer will generally be of short duration and highly localized, it may also be acceptable to use compounds which have some slight toxicity to these tissues. Moreover, surrounding and treatment sites may be protected with dark clothes, gloves, close shoes or caps to avoid unwanted photosensitizer activation between irradiation steps.

It is a further objective of the present invention to enhance the selectivity of the timed release photosensitizer towards unwanted species such as bacteria or fungi. The targeting is either achieved by chemically modifying the chemical or physical properties of the photosensitizer, or by coupling the molecule to a targeting moiety such as an antibody. It is generally preferred that the photosensitizer selected for use has a positive charge under physiological conditions since such photosensitizers are more readily taken up by the target microbes. Other targeting moieties that specifically recognize components of the bacterial cell membranes are known.

The present invention is further illustrated by the following examples, but is not limited thereby.

### Example 1

### Bacterial Reduction in Biofilms within the Oral Cavity

A dentist or patient can treat periodontal disease in various stages, or self-apply the treatment for prevention of diseases arising from microbes within the oral cavity. A composition containing photosensitizers encased in micro- or nanoparticles is administered either by a dentist or by the patient. For patient application, a mouthwash is used containing a high concentration of the nanoparticles. The patient takes a prescribed dose of the mouthwash, and applies it as one would apply traditional mouthwashes to coat the mouth. Patient would then wait a prescribed period, typically 4 to 6 hours, or apply the mouthwash before bedtime. During this time, the mouth's natural saliva would serve to flush most of the micro- or nanoparticles from the mouth, leaving only those particles which are associated with the biofilms or trapped in existing periodontal pockets. After the prescribed period, the dentist/patient would then administer radiation using a laser toothbrush (as seen in, for example, U.S. Pat. No. 5,658,148) to apply laser radiation for a prescribed duration or other means to direct irradiation to the treated sites within the oral cavity. The application would be similar in duration to the time taken to brush one's teeth. A prescribed frequency of about 2 times a day would effectively destroy harmful bacteria in the mouth. Merely to prevent recurrences a less frequent activation might be proposed of once a day or two.

### Example 2

### Periodontal Pocket Treatment for Disease

A dental practitioner applies the photosensitizer formulation. A gel containing the above-described micro- or nanoparticles is applied by the dentist directly to the periodontal pockets. This is preferable for patients with more advanced stages of periodontal disease, in that the dentist can insure that sufficient photosensitizer reaches the deeper pockets. In one preferred embodiment a gel is chosen which hardens after application, so that photosensitizer is set to slowly release over an extended period of time such as days/weeks etc. The pockets themselves should act to retain the photosensitizer for an extended period of time. The patient returns for periodic radiation treatments or in an alternative case self-applies radiation at prescribed intervals as described in Example 1 using a laser toothbrush, as mentioned above, or other suitable means.

### Example 3

### Fungal Reduction in Contaminated Toenail

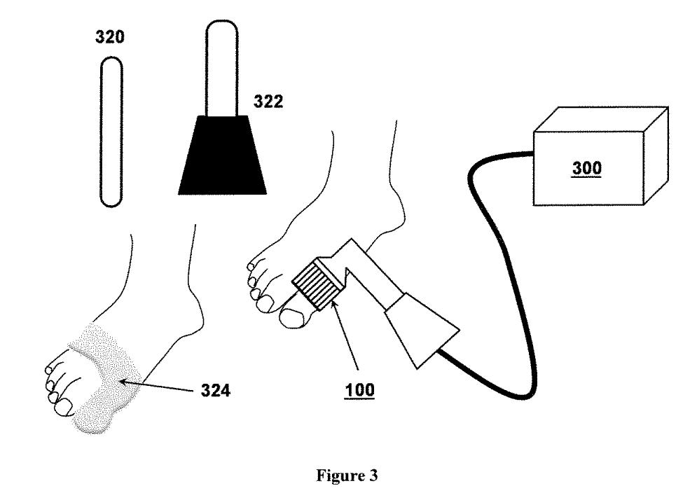

A health professional can treat onychomycosis in various stages, or patient can self-apply the treatment for prevention of diseases arising from fungi or yeast in the toenail. The toenail to be treated is “polished” with the aid of emery board 320 in order to reduce the nail thickness. A composition containing photosensitizers encased in micro- or nanoparticles is administered either by a health professional or by the patient. For patient application, lacquer 322 in dark bottle is used containing a high concentration of the nanoparticles. The patient covers the nail with a prescribed quantity of lacquer layers, and applies it as one would apply traditional nail polish to coat the nail. Then, patient would cover the infected toenail with dark material 324 to avoid premature activation of the photosensitizer. Patient would then wait a prescribed period, typically 4 to 6 hours, or apply the lacquer before bedtime. During this time, the micro- or nanoparticles would penetrate into the nail and release the photosensitizer. After the prescribed period, the health professional would then administer radiation for a prescribed duration using device 300, which provides laser radiation and oxygen, and delivery apparatus 100 to direct the appropriate radiation wavelength absorbed by the photosensitizer to the treated sites of the nail. A prescribed frequency of about twice a day would effectively destroy harmful fungi in the toenail. Merely to prevent recurrences a less frequent activation might be proposed of once every day, once every other day, or even once a week or month. (FIG. 3)

### Example 4

### Microbial Reduction in Contaminated Area of the Skin

A patient can self-apply the therapy for treatment or prevention of diseases arising from fungi or yeast in the skin such as chronic dermatophytosis. A composition containing photosensitizers encased in micro- or nanoparticles is administered by the patient. A spray, gel or lotion is used containing an appropriate concentration of the nanoparticles. The patient covers the contaminated area of scalp with a prescribed quantity of spray, gel or lotion and applies it as one would apply these traditional body products. Patient would then wait a prescribed period of time. Depending on the formulation the spray, gel or lotion may be applied before bedtime. During this time, the micro- or nanoparticles would release the photosensitizer which in turn would bind or attach to the pathogenic microbes. After the prescribed period, the patient would then administer radiation for a prescribed duration with the aid the laser device described in this invention with delivery apparatus 200. A determined frequency and laser radiation exposing duration would be prescribed depending on the nature of the skin-borne disease. To avoid uncontrolled photosensitizer activation, between radiation exposures the area treated is covered with dark materials such as a dark cap. Merely to prevent recurrences less photosensitizer formulation concentration might be proposed and undetermined laser radiation exposing duration.

### Example 5

### Disinfection or Sterilization of Manicure and Pedicure Tools

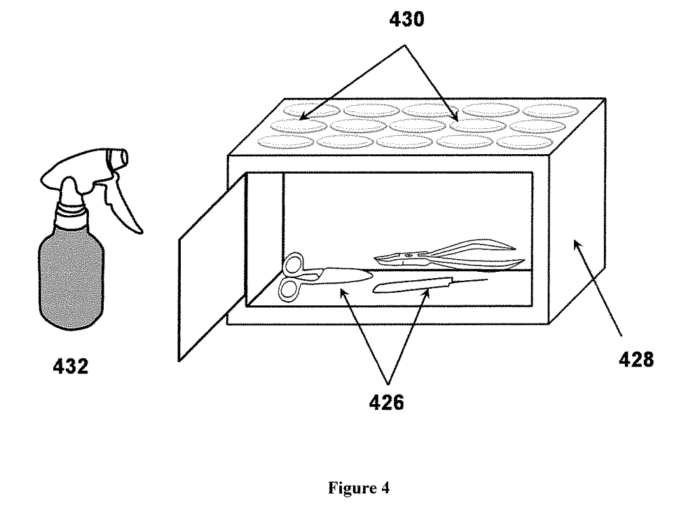

A health professional or nail technician can disinfect or sterilize fungi or yeast manicure and pedicure tools. Tools 426 to be disinfected or sterilized are placed inside device 428 like the one depicted in FIG. 4, capable of emitting an appropriate radiation wavelength absorbed by the photosensitizer by the aid of emitting diodes 430. A composition containing an appropriate concentration of photosensitizer encased in micro- or nanoparticles is applied on the surface of the pedicure and manicure tools with the aid of atomizer 432. Atomizer 432 is made of a dark colour material or is cover with a foil to avoid premature activation of the photosensitizer. Tools 426 are left inside device 428 during a determined period of time in which the micro- or nanoparticles would release the photosensitizer. Then, the professional would deliver radiation for a prescribed duration using emitting diodes 430 to direct the appropriate radiation wavelength absorbed by the photosensitizer to the manicure and pedicure tools.

Having described preferred embodiments of the invention, it is to be understood that the invention is not limited to the precise embodiments, and that various changes and modifications may be effected therein by those skilled in the art without departing from the scope or spirit of the invention as defined in the appended claims.

What is claimed is:

1. A method for the treatment of nail/skin microbial disease comprising the steps of:
a. administering a photosensitizer to an area needing treatment in a timed release formulation according to claim 12, wherein said timed release formulation controllably releases said photosensitizer to treatment sites;
b. allowing time for said time release formulation to release a portion of said administered dose; and
c. irradiating, at least twice, said treatment sites at preselected wavelengths to fight bacteria and fungi, once after step b and at least a second time after a predetermined time delay.

2. The method according to claim 1, wherein said irradiating step is applied repeatedly at predetermined intervals.

3. The method according to claim 2 wherein said predetermined intervals range for irradiating are from a period of several hours to several days.

4. The method according to claim 1 wherein said irradiating step uses preselected wavelengths, preferably between 630 and 800 nm.

5. The method according to claim 1, wherein said administering of said formulation is selected from a group consisting of direct application, application with a gel, application with a lacquer, application with a cream, application with a ointment, application with a sprayer, application with a lotion and combinations of them.

6. The method according to claim 1 wherein human toenail/skin are being treated.

7. The method according to claim 1, wherein manicure and pedicure tools are being treated.

8. The method according to claim 1 wherein said formulation includes micro- or nanoparticles that encase said photosensitizers, and wherein said timed release is achieved by controlled release of said photosensitizers from said micro- or nanoparticles.

9. The method according to claim 8, using particles which are biodegradable.

10. The method according to claim 8, wherein said timed release is achieved by periodically actively releasing said photosensitizers from a portion of said micro- or nanoparticles, wherein said method of actively releasing is selected from a group consisting of bulk erosion, radiation, ultrasonics, agitation through brushing, biochemical release, enzymatic release, chemical release and combinations of them.

11. The method according to claim 10, wherein said releasing by chemical release is by applying a solvent.

12. A composition for treatment of nail/skin microbial disease comprising a photosensitizer in a timed release formulation wherein said photosensitizer can only be partially activated by a first irradiation at preselected wavelengths to fight fungi and bacteria.

13. The composition according to claim 12, wherein said timed release formulation allows repeated irradiation at predetermined intervals over an extended time period without additional applications of said photosensitizer.

14. The composition according to claim 12 wherein said formulation includes nanoparticles that encase said photosensitizers, and wherein said timed release is achieved by the controlled release of said photosensitizers from said nanoparticles.

15. The composition according to claim 12 wherein said photosensitizers are photosensitive molecules, porphenes, selected from a group consisting of porphyrins, chlorins (dihydro-tetra phenyl porphyrins), bacteriochlorins (tetrahydro-tetra phenyl porphyrins, pheophorbides, bacteriopheophorbides, and phthalocyanines.

16. The composition according to claim 12 are wherein said photosensitizers are known photosensitizer precursors, which naturally convert to a photosensitizer upon administration to a patient.

17. The composition according to claim 16, wherein said photosensitizer precursor is aminolevulinic acid.

18. The composition according to claim 12 wherein said preselected wavelengths are in the range of about 630 nm to about 800 nm.

19. The composition according to claim 12 wherein said photosensitizer comprises micro- or nanoparticle complexes to achieve high concentrations of said photosensitizer at a treatment area.

20. The composition according to claim 19, wherein said micro- or nanoparticle complexes are dendrimer-photosensitizer complexes.

21. The composition according to claim 12, wherein said photosensitizer comprises a modified photosensitizer complex which selectively targets unwanted species of bacteria and fungi, while leaving other species relatively intact.

22. The composition according to claim 21, wherein said modified photosensitizer complex to selectively eliminate unwanted species has antibodies attached to said photosensitizer complex.

23. The composition according to claim 21, wherein said modified photosensitizer complex to selectively eliminate unwanted species has molecular fragments, which target components of fungal/bacterial cell membrane, attached to said photosensitizer complex.

24. A device for the treatment of nail/skin fungal disease comprising:
a. at least one radiation source operating at a radiation wavelength preselected from the range of about 630 and 800 nm; and
b. a light delivery apparatus comprising at least one optical fiber and internal conduit.

25. The device according to claim 24, wherein said radiation source includes coherent and incoherent radiation sources selected from a group consisting of laser sources, light emitting diode sources, lamp radiation sources, sunlight and combination of them.

26. The device according to claim 24, wherein said internal conduit accommodates a substance selected from a group consisting of extra oxygen in gaseous state, oxygenated solution, solution containing the photosensitizer, and combinations of them.
